(19) United States
(12) Patent Application Publication
(10) Pub. No.: US 2023/0091429 A1
(43) Pub. Date: Mar. 23, 2023

(54) COMPOSITION FOR ENHANCING CHANCES OF CONCEPTION AND FEMALE SEX RATIO IN BOVINES

(71) Applicant: AGROCLEAN, Vadodara (IN)

(72) Inventors: Vikas GUPTA, Chandigarh (IN); SudhanshuKumar Ranjitkumar SINGH, Vadodara, Gujarat (IN)

(21) Appl. No.: 17/909,366

(22) PCT Filed: Mar. 5, 2021
(86) PCT No.: PCT/IN2021/050214

§ 371 Date: Sep. 3, 2022

(30) Foreign Application Priority Data
Mar. 5, 2020 (IN) 201921035828

(51) Int. Cl. A61K 31/12; A61K 47/26; A61K 47/18; A61K 9/00; A61K 31/197; A61P 15/08

(52) U.S. Cl. CPC A61K 31/12; A61K 47/26; A61K 47/183; A61K 9/0053; A61K 31/197; A61P 15/08

(57) ABSTRACT

The present invention includes an economic, synergistic, non-toxic and stable oral composition comprising curcuminoid or its derivative, polyhydric alcohol, amino acids, buffering agent and other pharmaceutically acceptable excipients for enhancing the female sex ratio and improved chances of conception in Bovines. The oral composition is acidic in nature with pH between 4.1-4.6 and in semi-viscous form to enable the availability of the composition in buccal cavity for at least 2-3 minutes. The present invention also provides an economic process for the preparation of said composition.

### FIELD OF THE INVENTION

The present invention pertains to dairy, in particular to a composition for application in dairy Bovine animals to alter sex ratio and enhance chances of producing more number of females, and the process for its preparation. The alteration in the sex ratio takes place in vivo and through oral administration.

### BACKGROUND

Dairy industry is one of the fastest growing industries in the world and it has reached a value of US$ 673.8 billion in 2018. Dairy industry includes any business enterprise that deals with the processing and harvesting of animal milk obtained from cow, buffalo, goat, camel and sheep for human consumption. Demand of livestock such as cattle, buffalo, sheep and goat are increasing in accordance with the demand of their products. This is due to the increased awareness about the importance of protein in food, besides the increase in disposable income and education. Further, the demand for specific sex of livestock depending on the requirement like milk or meat is also important. Whereas Farmers tend to raise male livestock for meat as they relatively grow faster. Dairy farmers prefer to have determination of calf sex in advance.

Dairying has become an important secondary source of income for millions of rural families and has assumed the most important role in providing employment and income generating opportunities, particularly for marginal and women farmers. Most of the milk is produced by animals reared by small, marginal farmers and landless laborers. According to the National Action Plan for Dairy Development—VISION 2022 By DADF-Government of India, of the total milk production in India, about 48% milk is either consumed at the producer level or sold to non-producers in the rural area. The balance 52% of the milk is marketable surplus available for sale to consumers in urban areas.

The milk can be consumed directly or in the form of yogurt, cheese, ice cream, butter and condensed milk etc. It contains various important nutrients such as calcium, vitamin D and B-12, protein etc. and micronutrients like zinc or magnesium etc. and therefore it is essential for human health. With increasing demand for dairy products in respect of feeding a growing population and increasing health consciousness, the dairy industry at present is facing a serious problem to meet the current demand due to the presence of large number of unproductive male cows and poor fertility rate in female cows.

To meet the increasing demand for dairy product in India and other countries, the dairy industry has been continuously trying various cross-breeding techniques and other methods to increase the milk production. However, developing an alternative method/system whereby the milk production can be increased by producing large number of female-offspring without causing any change in the genetic profile of the off-spring is one of the approaches to solve the present problem.

In Indian culture where cow is worshipped as goddess, merely less than 40% of India's cows only are productive. This coupled with high milk demand renders the male calf not productive and are mostly left unattended.

### Scenario of Dairy Industry in India

Currently India is the world's largest producer and consumer of milk accounting for 19% of the world milk production and consisting of about 57% of world buffalo and 16% of world cattle population. According to the livestock census 2012, the country has 133.2 million adult female cattle and buffalo and 88.35 million in milk cattle and buffaloes. The average productivity of in milk cattle and buffalo in the country is about 4.65 KgPD (2015-16) which is far below as compared to the productivity levels of In-milk animals in dairy developed nations.

India has the largest number of livestock (300 million), including buffalo, and the largest number of dairy farming households (75 million) in the world. India ranks first among the world's milk producing nations since 1998 and has the largest bovine population in the World. Milk production in India during the period 1950-51 to 2016-17, has increased from 17 million tonnes to 163.7 million tonnes recording an annual growth of 6.65%. FAO reported 1.46% increase in world milk Production from 800.2 million tonnes in 2016 to 811.9 (Estimated) million tonnes in 2017. The per capita availability of milk in the country which was 130 gram per day during 1950-51 has increased to 352 gram per day in 2016-17 as against the world estimated average consumption of 294 grams per day during 2017. This represents sustained growth in the availability of milk and milk products for our increasing population.

More so, India being the world's largest milk producing countries with a market value of INR 9,168 Billion, suffers due to low milk production. The amount of milk produced per cow in India is considerably behind the amount of milk produced per cow in other countries. This could be due to the longer time taken by cows to become fertile, presence of large number of unproductive male cows, poor quality of feed and fodder, reduced forage area comprising grass, poor management practices, natural calamities and variable weather conditions.

### SOLUTION TO THE PROBLEM

### Current Prevailing Solutions

The sex ratio between males and females in bovine or animals as referred in this application (cattle, including cows, oxen, goats, sheep, bison, buffalo and other animals generally referred as cattle) is more or less equal. However, in diary producing animals, it is beneficial to have female off springs in a higher ratio that the males. There has been already report in increasing the chances of conception rate and also to increase the number of births to female offspring in mammals particularly in bovines involving various technology such as sexed semen technology, sex fixing technology or by using composition comprising certain ingredients which helps in conception or to increase female offspring.

U.S. Pat. No. 5,110,592 discloses a method for increasing the number of live births to a female animal, wherein the mother animal is fed with an effective amount of an edible composition comprising ground flaxseed at least one week before the expected time of birth. The disclosed flaxseed composition can be admixed with a suitable animal feed. The flaxseed composition may further comprise zinc or vitamin B-6 or both zinc and vitamin B-6. However, there is no evidence of the utility and industrial application of the sample.

U.S. Pat. No. 7,351,581 discloses an in vivo method for producing female off-springs in bovines by administering a therapeutically effective amount of composition comprising acetic acid (approximately 8-16% dilute acetic acid), or its pharmaceutically acceptable derivatives in the range of 250-400 mL to female bovines just after or before insemination. The disclosed composition also comprises vinegar, sodium acetate and/or their natural or synthetic derivatives. The problem in using the composition to produce female off-springs is that the composition is highly volatile, has very short half-life period and possibility of irritation problem in the muzzle and buckle may be due to the presence of acetic acid and therefore making the composition not suitable for use in market. The composition requires additional non-irritating excipients or carriers to overcome this issue.

U.S. Pat. No. 9,107,402 discloses a composition and a method to modify the sperm fertility and sex ratio of mammalian offspring, wherein the composition comprising an amount of a class of compounds of phenoxazine or phenothiazine derivatives in addition with an electron transfer agent capable of oxidizing a reduced cofactor (NADPH or NADH) and an amount of a hexose sugar and/or the phosphorylated esters of such sugars. When the composition is mixed with living sperm prior to or at the time of insemination, it results in modified rates of conception and an alteration of birth sex ratio. The drawback associated with the disclosed composition is, the composition can be stabilized only by lyophilizing it at 0° C. to 15° C. using a lyophilizer equipment, which is expensive and not industrially viable.

A M Noorlander et al (Reproductive Bio-Medicine Online (2010) 21, 794-802) discloses a natural sex selection method i.e. natural female gender pre-selection method through maternal diet in combination with timing of sexual intercourse. It describes further that maternal diet low in sodium and high in calcium in combination with timing of intercourse well before ovulation can improve the chances of conceiving a girl. A success rate of percentage of delivery of female child is 81% but it requires correct maternal diet along with correct timing of intercourse and regular monitoring of mineral content in the blood to achieve the birth of female child.

G. E. Seidel (Animal, May 2014, pages 160-164) discloses an update on sexed semen technology wherein the technology involves the use of flow cytometer/cell sorters which can make over 30000 consecutive evaluations of individual sperm each second for each nozzle and sort the sperm into three containers: X-sperm, Y-sperm and un-sexable plus dead sperm. Even at these speeds it is not economical to package sperm at standard numbers per inseminate. The pregnancy rates in cattle with 2 million sexed sperm per insemination dose are about 80% of those with conventional semen at normal sperm doses. This lowered fertility could be due to damage to sperm while sorting. In addition to this issue, the extra cost of sexed semen limits the applications that are economically feasible. In spite of abovementioned problems, 2 million doses of bovine semen are sexed annually in the United States.

The main application of the above-mentioned sexed semen technology is predominantly for dairy heifers to have heifer calves for the purpose of herd expansion, sale as replacements or often for eventual export. Breeders of purebred cattle often use sexed semen for specific mating, thawing and then sexing frozen semen and immediately using the few resulting sexed sperm for in vitro fertilization. Beef cattle producers are also starting to use sexed semen to produce crossbred female replacements. In spite of its potential application in dairy industry, sexed semen technology suffers because of the cost associated with the instrument used to perform the technology such as cytometer etc and poor fertility rate.

Waleed F. A. Marei et al. (International Journal of Veterinary Science and Medicine 6 (2018) S36-S40) discloses the both dietary and in-vitro supplementation of omega 6 PUFAs (polyunsaturated fatty acid) affect the sex ratio of the offspring in cattle, particularly towards the birth of male offspring. If further discloses that sex ratio is skewed to the male side in the embryos derived from omega 6 linoleic acid (LA) and trans-10, cis-12 conjugated linoleic acid (CLA) treated oocytes (79% and 71% respectively) compared to control and omega-3 alpha-linolenic acid (ALA) treated oocytes (44% and 54%, respectively).

Thus, in order to overcome the problems existed in the prior art references such as inclusion of additional non-irritating agents, use of costly instrument for example lyophilizer or flow cytometer etc and poor conception rate, there is an urge to provide a stable, non-toxic, effective, economic and synergistic composition for enhancing the birth of female offspring without affecting the genetic profile and also for increasing the conception rate in bovines without side effects to the off-spring and without any leaching in the milk produced by the bovine.

### OBJECT OF THE INVENTION

The object of the present invention is to provide an oral composition for improving chances of conception and enhancing the female sex ratio in Bovines and a process for producing the same and uses of the composition.

### SUMMARY OF THE PRESENT INVENTION

The present invention provides a novel and synergistic oral composition that provides enhancement in conception ratio and enhancing the female sex ratio in bovines. The oral composition comprising of curcuminoid or its derivative, polyhydric alcohol, amino acids, and buffering agents along with other Pharmaceutically acceptable excipients. The composition particularly comprising Demethoxycurcumin 5-10%, sorbitol 10-30% as a carrier, Amino acids (aspartic acid 1-2% and glutamic acid 1-2%) as stabilizers, Acetates/citrate as buffering agents 12-16% and pharmaceutically acceptable excipients 38-52%. The composition is essentially with acidic pH and semi-viscous in nature. The composition increases the birth of female calves ranging from 78-80% and cumulatively to 79%. The oral composition of the present invention also enhances the conception rate in dairy animals provided the standard procedures of the product administration are followed strictly. The present invention also provides a simple and economic process for the preparation of the said oral composition.

### DETAILED DESCRIPTION OF THE PRESENT INVENTION

Demand for the dairy products in India and other countries is expected to increase in the coming years driven by more consumers, increase in income and health consciousness. With poor fertility in cows and increasing number of unproductive male cows, it may not be possible to meet the global demand for the dairy products. There is a necessity in developing an alternative method to increase the milk production by enhancing the conception rate and also by altering the sex ratio to especially female offspring in the bovines without causing any change in the genetic profile of the offspring.

The present invention provides a simple, economic, safe, efficient and synergistic oral composition including curcuminoid or its derivative, polyhydric alcohol, amino acids, or its derivative, buffer agents and other pharmaceutically acceptable excipients to achieve the enhancement in the percentage delivery of female offspring and in the conception rate in bovines.

The oral composition of the present invention particularly comprises of Demethoxycurcumin in the range of 5-10%, Sorbitol in the range of 10-30%, amino acids in the range of 2-4%, buffering agent in the range of 12-16% and other excipients including water in the range of 48-62%.

The curcuminoid included in the present composition is selected from a group consisting of curcumin or its derivative, preferably selected from demethoxycurcumin.

The polyhydric alcohol in the present composition is selected from the group consisting of pentaerythritol, xylitol, adonitol, arabitol, mannitol, sorbitol, dulcitol or other carbohydrate based polyhydric alcohol, preferably the polyhydric alcohol is selected from Sorbitol. The polyhydric alcohol plays a very important role of the carrier in the product delivery system.

The amino acids used in the present composition are essentially to act as stabilizers and are selected from a group consisting of glycine, alanine, valine, leucine, isoleucine, phenylalanine, aspartic acid, glutaric acid, asparagine, glutamine, arginine, lysine, cysteine, threonine, tyrosine, proline, histidine, tryptophan, methionine and serine or combination thereof, preferably the amino acid is selected from glutamic acid, aspartic acid or combination thereof. The percentage range of amino acid in the composition can be varied between 1-2% individually and together 2-4%.

The buffering agents of the present composition may be selected from buffer agent, which can provide acidic pH such as citrate buffer or acetate buffer so as to regulate the pH of the composition between 3.5 to 5.5, preferably 4 to 5, most preferably 4.1-4.6.

The other excipients used in the present invention for making the composition suitable for administration in bovines may be selected from binder, stabilizing agent, flavouring agent, sweetening agent, viscosity modifying agent, water or any other compound which is not disturbing the pH of the resulted final composition material.

The composition of the present invention exhibits high stability at varied temperature range between 6-25° C. and hence, it requires no special arrangement/instrument like lyophilizer etc for storage. The composition is particularly in semi-viscous form so that it becomes easy for the subjected animal to retain the composition for at least 2-3 minutes in the Buccal cavity for chewing purpose and mixes well with the saliva. By retaining the composition for 2-3 minutes in the buccal cavity, flow rate of saliva is increased by 10-12 times and it remained higher 2.7 times more when compared to the control group. Further, the pH of the saliva is also dropped to 4.7 from 7.2 and remained at 4.7 for more than 96 hours. The increased saliva rate and drop in pH condition assist in creating favourable condition which facilitates the conception possibility in the bovines and particularly towards the birth of female offspring.

The composition of the present invention:
i. exhibits high stability at varied temperature range between 6-30° C.
ii. is semi-viscous form and retained for 2-3 minutes in the Buccal cavity.
iii. mixes with the saliva in 5 to 6 minutes, flow rate of saliva is increased by 10-12 times and it remained higher 2.7 times more when compared to the control group.
iv. the pH of the saliva is also dropped to 4.7 from 7.2 and remained at 4.7 for more than 96 hours.

The efficacy of the composition of the present invention is studied in bovines including Heifers and its subsequent parturition. The percentage delivery of female offspring is increased from 45% obtained in the control group to 80% and even in 2^{nd} or 3^{rd} parity, Heifers administered with the present composition showed equally good results. The conception rate is also found to have enhanced with the application of the present composition by following the standard protocol of the product administration.

The composition of the present invention is safe and non-toxic and showed no side effects such as gastrointestinal diseases, uneasiness, fever, skin rashes etc. in the mother animal or in the female offspring produced. More importantly, there is no genetic disorder observed in both mother animal and female offspring produced.

The present invention also discloses a process for the preparation of the present composition, wherein the said process comprising the steps of:
i. Mixing of Amino acid and curcuminoid or its derivative with water to prepare a mixture.
ii. Mixing of both polyhydric alcohol and buffer agent in water to obtain a mixture.
iii. Blending of mixtures from step (i) and (ii) together with pharmaceutically acceptable excipients.
iv. Evaporating excess water under vacuum to yield the composition in the semi-viscous form.

The process parameters including temperature and pressure which are room temperature and normal atmospheric pressure.

The process for the preparation of oral composition of the present invention is carried out in an aseptic environment to avoid any contamination in the prepared composition or to avoid any infection or inflammation in the animal treated with the present composition. The pH of the composition is maintained between 4.1-4.6.

The present invention also includes various dosage forms of the composition other than the desirable semi-viscous form. For example, the composition material can be in the form of powder, tablet, dispersible tablets or liquid etc. The route for administration of the disclosed composition is basically depends on the subject. In the present invention, subject being the bovine, the composition may be administered via oral administration, particularly through Buccal cavity. Other routes of administration of the present composition may include intravenous, cutaneous, sub-cutaneous, parenteral, vaginal, intra-vaginal, anal routes etc. The composition may be applied either directly or formulated in any desired physical forms.

The present invention further describes about the dose of administration of the oral composition which may vary according to the body weight of the bovine animal. The prescribed dose for mammal is generally between 0.2-1.0 g/kg body weights of the subjected animal. Accordingly, the amount of present formulation to be given to cows and buffaloes with body weight of 400-600 kg may vary between 250-300 Gms. The application of a very high dosage of composition may lead to lethal effect or serious side effect to the animal.

Apart from describing suitable dosage form, routes for administration and dosage quantity of the present composition, the present invention further discloses the perfect time for administration of composition, which is crucial for bringing out the better result of the composition i.e. 30 minutes before insemination or at least within one hour after insemination. If the material is administered after this prescribed crucial time, then the composition ingredients may not be available to the animal before ovulation and may not produce the desired result of birth of female offspring.

In yet another embodiment, the present invention relates to a method of increasing sex ratio in bovine said method comprising orally administering to the bovine once the composition of the present invention, during 24 to 48 hours before natural or artificial insemination in a dose of between 0.2-1.0 g/kg body weights of the subject bovine.

Accordingly, the amount of present formulation to be given to cows and buffaloes with body weight of 400-600 kg may vary between 250-300 g. It is envisaged that a dose up to 320 gms can be administered to a bovine of 600 Kg.

The significance of having specified components in the oral composition is as follows:
1. Presence of polyhydric alcohol preferably Sorbitol increases the flow rate of saliva because of its chewing in the Buccal cavity which helps in reducing pH of the saliva from 7.2 to 4.7 and thus provides favorable conditions for conception and to the birth of female offspring in bovines.
2. Curcuminoid or its derivative, preferably Demethoxycurcumin acts as an anti-inflammatory agent to prevent any inflammation and infection in the administered animal. It also acts as an immunomodulator and helps animal to have healthy and infection free reproductive organs and zygote formation site without affecting the pH.
3. Buffer agent helps in regulating and maintaining the pH of the composition between 4.1-4.6.
4. Presence of amino acid (glutamic acid and aspartic acid) to an extent helps as a stabilizer and also in creating an environment that will create negatively charged ovum to attract positively charged X chromosome bearing sperm and thus producing XX zygote i.e. a female.
5. Ingredients used in the composition are stable and safe and therefore the composition does not require any costly equipment for storage such as lyophilizer and shows no side-effect.

The following examples are given as particular embodiments of the present invention and to demonstrate the practice and advantage thereof. It is to be understood that the examples are given by way of illustration and are not intended to limit the specification or the claims that follow in any manner.

### EXAMPLE 1

### Composition C1-C5 of the Present Invention

Various illustrative compositions of the present invention are provided at Table 1 below:

TABLE 1
Composition of the present invention

| Ingredients | C1 | C2 | C3 | C4 | C5 |
|---|---|---|---|---|---|
| Demethoxycurcumin | 8 | 5 | 7 | 9 | 10 |
| Sorbitol | 22 | 15 | 30 | 20 | 10 |
| Aspartic acid | 2 | 1 | 1 | 2 | 1 |
| Glutamic acid | 2 | 1 | 2 | 1 | 1 |
| Buffering Agent | 16 | 16 | 12 | 12 | 16 |
| Total Active Ingredients | 50 | 38 | 52 | 44 | 38 |
| Others & Water | 50 | 62 | 48 | 56 | 62 |
| Total | 100 | 100 | 100 | 100 | 100 |

### EXAMPLE 2

### General Procedure for the Preparation of C1-C5 Composition of the Present Invention

The C1-C5 composition of the present invention is prepared by mixing the amino acids, demethoxycurcumin and water to obtain a first mixture. Then, sorbitol and suitable buffering agent are mixed with water to get a second mixture. Both mixtures are blended together with pharmaceutically acceptable excipients followed by the evaporation of excess water to obtain the formulation in the desired dosage form for oral administration. The pH of the resulting material is maintained between 4.1-4.6 with the use of suitable buffer agents. The process is generally carried out in an aseptic environment.

### EXAMPLE 3

### Preparation of Composition of the Present Invention

Aspartic acid (2.0 g), glutamic acid (2.0 g) and Demethoxycurcumin (8.0 g) are mixed well with water (20.0 g) to get a first mixture. Then, Sorbitol (22.0 g), citrate buffer (16.0 g) and water (20.0 g) are mixed together to prepare a second mixture. Both mixtures are blended together with maltose excipients (5.0 g) and glycerin (5.0 g), concentrated under vacuum to remove excess water to obtain the composition in a semi-viscous form.

### EXAMPLE 4

### Preparation of Composition of the Present Invention

Aspartic acid (1.0 g), glutamic acid (2.0 g) and Demothoxycurcumin (7.0 g) are mixed well with water (20.0 g) to get a first mixture. Then, Sorbitol (30.0 g), acetate buffer agent (12.0 g) and water (20.0 g) are mixed together to prepare a second mixture. Both mixtures are blended together with maltose excipient (5.0 g) and glycerin (3.0 g), concentrated under vacuum to remove excess water to obtain the composition in a semi-viscous form.

### EXAMPLE 5

### Effect of the Components of the Present Invention

Several compositions were made by varying the components of the present invention and the same are elaborated in Table 2 below:

TABLE 2
Testing of importance of the composition of the present invention

| Ingredients | F1 (C1) | F2 | F3 | F4 | F5 |
|---|---|---|---|---|---|
| Demethoxycurcumin | 8 | 0 | 7 | 9 | 10 |
| Sorbitol | 22 | 20 | 0 | 20 | 10 |
| Aspartic acid | 2 | 1 | 1 | 0 | 1 |
| Glutamic acid | 2 | 1 | 2 | 0 | 1 |
| Buffering Agent | 16 | 16 | 12 | 15 | 0 |
| Total Active Ingredients | 50 | 38 | 82 | 44 | 54 |
| Others & Water | 50 | 62 | 48 | 56 | 62 |
| Total | 100 | 100 | 100 | 100 | 100 |

The F1 is per the composition C1 of the present invention. The compositions F2 to F5, miss one of the essential components of the composition. The above table clearly shows the interaction between the essential ingredients to provide the surprising efficacy of the components of the composition and shows the deleterious of effect of altering the components of the composition.

The compositions F1 to F5 were tested by Veterinarians in the field. It was found that the composition F1 was the only active composition. The compositions F2 to F5 were not formed into a stable mixture or were not active. F1 also gave the best results as the availability all the ingredients ensured better and consistent results. The absence of Demethoxycurcumin in F2 resulted in the inconsistent uterine pH. The absence of Sorbitol in F3 had lesser stability and was not available in the buccal cavity for longer duration. The absence of the mentioned amino acids in F4 resulted in lesser motility of the sperms in the uterus. The total absence of the Buffering agents in F5 did not provide the desired pH.

It has been observed that when there is change in the physicochemical parameters, there is a change in efficacy and only the composition Cl of the present invention. From the above it can be clearly seen that unless all components of the composition are present the desired results of altering the sex ratio cannot be achieved. In other words, the composition is synergistic showing unexpected efficacy by the interaction of its ingredients. This composition and its efficacy are arrived after much human efforts and several clinical trials.

### EXAMPLE 6

### Activity of the Composition of the Present Invention

The efficacy of the present composition in enhancing conception ratio and female sex ratio in diary animals is tested on initial trial basis in bovine animals. During the trial testing, the composition is administered as per the standard protocol and in the presence of supervision of qualified Veterinarians, Breeders and Applicators. The results are tabulated in Table 3.

TABLE 3
Effect of the composition of the present invention
Table 3A: Effect in Heifers

| Heifers | With new Composition | | Controlled | |
|---|---|---|---|---|
| AI (Artificial | 28 | | 25 | |
| insemination) | | | | |
| Pregnancy | 20 | | 11 | |
| Conception Ratio | 71.43% | | 44.00% | |
| Female | 16 | 80.00% | 5 | 45.45% |
| Male | 4 | 20.00% | 6 | 54.55% |

The birth of female offspring in case of heifers administered with the new composition is up-to 80% compared to 45.45% in the controlled group. The conception ratio of heifers subjected to present composition is also improved to 71.43% from 44%.

TABLE 3B
Effect in Animals in 2-3 parity

| 2^{nd} -3^{rd} Parity | With new Composition | | Controlled | |
|---|---|---|---|---|
| AI | 35 | | 40 | |
| Pregnancy | 23 | | 19 | |
| Conception Ratio | 65.71% | | 47.50% | |
| Female | 18 | 78.26% | 9 | 47.37% |
| Male | 5 | 21.74% | 10 | 52.63% |

The birth of female offspring in case of 2^{nd} and 3^{rd} Parity administered with the new composition is up-to 78.26% little lesser in comparison to Heifers and compared to controlled group it is much higher at 47.37%. The conception ratio of heifers subjected to present composition is also improved to 65.71% from 47.50%.

TABLE 3C
Effect in Heifers & Animals in 2nd-3rd parity

| Total AI | With new Composition | | Controlled | |
|---|---|---|---|---|
| AI | 63 | | 65 | |
| Pregnancy | 43 | | 30 | |
| Conception Ratio | 68.25% | | 46.15% | |
| Female | 34 | 79.07% | 14 | 46.67% |
| Male | 9 | 20.93% | 16 | 53.33% |

Table 3B and 3C clearly indicate that the composition of the present invention is quite effective in improving the chances of producing female off-springs even in 2^{nd} or 3^{rd} parity animals including Heifer animals in 2^{nd} or 3^{rd} parity. The percentage of delivery of female calves in Heifers and animals in 2^{nd} or 3^{rd} parity cumulatively is around 79.07% compared to 46.67% via controlled method. The conception rate is also increased up to 68.25% with the application of the composition of the present invention compared to 46.15% achieved via controlled method.

The toxicity effect of the present composition is also studied in bovines. The composition is found to be safe and non-toxic and showed no side-effects such as gastrointestinal disorder, genetic disorder, increase in body temperature and uneasiness etc. in both subjected animal and its offspring.

### Advantages Of The Present Composition

Composition is highly palatable product with soothing flavor.

Composition shows better stability at a temperature range between 6-25 ° C.

Composition can be easily metabolized and thus eliminates chance of any genetic disorder.

Composition may be administered just on the onset of the heat of the animal.

Composition provides balanced pH at the zygote formation site and stability for enhancing female ratio in Bovines.

It provides no side effects on mammals after the application.

TABLE 4
Effect of the individual Components in the
composition of the present invention

| Ingredient | Activity |
|---|---|
| Sorbitol | 10% |
| Aspartic acid | 3% |
| Glutamic acid | 3% |
| Acetic acid | 23% |
| Acetate buffer | 21% |
| Demethoxycurcumin | 5-7% |
| Maltose | Nil |
| Composition of the present invention | 83% |

From the above table it is very clear that the addition of the active ingredients like Aspartic acid and Glutamic acid have some effect on the performance of the product by supplementing the energy to the sperms. The presence of the derivatives of Curcumin Demethoxycurcumin ensures better health to the animal and also provides immune-modulatory benefits to the animal. The presence of Demethoxycurcumin also provides better motility to the sperms. The entire composition helps to maintain the pH of the Reproductive organs to the acidic as the post ovulation pH of the Ovum is more likely alkaline and the uterine pH is expected to be 6.7 to 7.2. The present composition ensures to have a negatively charged environment thus enabling the higher chances of having XX zygote formation.

### EXAMPLE 7

### Effect of the Composition of the Present Invention in Female Ratio in Cows

The composition of the present invention of Polyhydric Alcohols, Amino acids, Maltose, Demethoxycurcumin and buffers of acetates as illustrated at tables 5 and 6 below was tested for its efficacy in cows (Table 5) and in Buffaloes (Table 6) after obtaining necessary ethical clearances. The composition was administered at a dose of 320 grams per animal single dose of the composition at the on-set of heat symptoms, i.e. at the onset of oestrous cycle.

TABLE 5
Effect of the composition of the present invention on cows

| | | | Number | | | |
|---|---|---|---|---|---|---|
| | | | of animals | | | |
| | | Number | with | | Sex of new- | |
| Sr. | | of animals | Confirmed | Calving | born calf | |
| No. | Group No. | in group | pregnant | occurred | Female | Male |
| 1 | Group-1 | 40 | 25 | 23 | 19 | 4 |
| | Percentage | | 62.5 | 92 | 83 | 17 |
| 2 | Group-2 | 40 | 26 | 25 | 20 | 5 |
| | Percentage | | 65 | 96 | 80 | 20 |
| 3 | Group-3 | 40 | 21 | 18 | 16 | 2 |
| | Percentage | | 52.5 | 85.7 | 89 | 11 |
| 4 | Group-4 | 40 | 18 | 15 | 14 | 1 |
| | Percentage | | 45 | 83 | 93 | 7 |
| 5 | Group-5 | 40 | 21 | 19 | 15 | 4 |
| | Percentage | | 53 | 90 | 79 | 21 |
| 6 | Group-6 | 40 | 32 | 28 | 23 | 5 |
| | Percentage | | 80 | 87.5 | 82 | 18 |
| 7 | Controlled | 40 | 16 | 15 | 8 | 7 |
| | Group | | | | | |
| | Percentage | | 40 | 93.75 | 53.33 | 46.67 |

TABLE 6
Effect of the composition of the present invention on Buffaloes

| | | number of | | | |
|---|---|---|---|---|---|
| | Number | animals | | | |
| | of | confirmed | Calving | sex of new-born calf | |
| Group No. | animals | Pregnancy | occurred | Female | Male |
| Group-1 | 40 | 20 | 19 | 17 | 2 |
| Percentage | | 50 | 95 | 89 | 11 |
| Group-2 | 40 | 23 | 23 | 19 | 4 |
| Percentage | | 58 | 100 | 83 | 17 |
| Group-3 | 40 | 15 | 15 | 13 | 2 |
| Percentage | | 38 | 100 | 87 | 13 |
| Group-4 | 40 | 21 | 19 | 17 | 2 |
| Percentage | | 53 | 90 | 89 | 11 |
| Group-5 | 40 | 15 | 14 | 13 | 1 |
| Percentage | | 38 | 93 | 93 | 7 |
| Group-6 | 40 | 19 | 18 | 16 | 2 |
| Percentage | | 48 | 95 | 89 | 11 |
| Controlled Group | 40 | 15 | 14 | 7 | 7 |
| Percentage | | 37.5 | 93.33 | 50 | 50 |

From Tables 5 and 6 as above, it can be seen that the Composition in both Cows and Buffaloes demonstrates enhanced conception and an increased female ratio of 80% on various parameters with the ratio of the female calves as high as 93% in comparison to the controlled group where the conception was as low as 37.5% in Buffaloes and 40% in cows and the female ratio in Buffaloes was 50% and in cows it was 53.3%.

### EXAMPLE 7

### Comparison of the Composition of the Present Invention with Prior Art Composition

The composition of the present invention is compared with a prior art composition and other compositions such as in Aulprofem and Ratio, and the results are provided as here below at Table 7.

TABLE 7
Showing the comparison of the composition of the present invention with prior art
composition

| P1 | | P2 | | | |
|---|---|---|---|---|---|
| (Aulprofem) | | (Ratio) | | Present composition (C1/F1) | |
| Acetic Acid | 10% | Sorbitol | 64% | Demethoxycurcumin | 8% |
| Sodium Acetate | 6% | Aspartic acid | 0.5% | Sorbitol | 22% |
| | | Glutamic acid | 1.5% | Aspartic acid | 2% |
| | | Acetate buffer | 4.0% | Glutamic acid | 2% |
| | | Maltose | 15% | Buffering Agent | 16% |
| | | Others & Water | 15% | Total Active Ingredients | 50% |
| | | Total | 100% | Others & Water | 50% |
| | | | | Total | 100% |

### The Composition of the Above-Mentioned Invention is Better Than the Two Previous Ones on Various Parameters

The composition is more stable and consistent due to stability of pH.

The conception also increased considerably using the composition of the current invention.

The presence of Curcuminoid or its derivatives ensures the healthy uterine and other reproductive organs as its takes care of any possibility of the infection at the zygote formation site and also acts a strong immune-modulator.

The overall female births also improved with the current invention.

Overall results with the current composition of the new invention were better than the previous two.

From the above and based on clinical trials, it can be seen that the composition of the present invention has enhanced efficacy than prior art compositions and provides better female sex ration in comparison to other compositions of prior art known for such purposes.

1. A synergistic oral composition for enhancing chances of conception and female sex ratio in bovines, wherein the composition comprising curcuminoid or its derivative, polyhydric alcohol, amino acids, or its derivative, buffer agents and other pharmaceutically acceptable excipients.

2. The synergistic oral composition as claimed in claim 1, wherein the composition comprises Demethoxycurcumin in the range of 5-10%, Sorbitol in the range of 10-30%, amino acids in the range of 2-4%, buffering agent in the range of 12-16% and other excipients including water in the range of 48-62%.

3. The synergistic oral combination as claimed in claim 1, wherein the polyhydric alcohol is selected from a group consisting of pentaerythritol, xylitol, adonitol, arabitol, mannitol, sorbitol, dulcitol or other carbohydrate based polyhydric alcohol, preferably the polyhydric alcohol is selected from Sorbitol in the range of 10-30%.

4. The synergistic oral combination as claimed in claim 1, wherein the composition comprises stabilizers selected from a group consisting of glycine, alanine, valine, leucine, isoleucine, phenylalanine, aspartic acid, glutaric acid, asparagine, glutamine, arginine, lysine, cysteine, threonine, tyrosine, praline, histidine, tryptophan, methionine and serine or combination thereof, preferably the amino acid is selected from glutamic acid, aspartic acid or combination thereof, the percentage of 1-2% individually and together 2-4%.

5. The synergistic oral combination as claimed in claim 1, wherein the buffer is selected from citrate buffer or acetate buffer and pH of the composition ranges from 3.5 to 5.5, preferably 4 to 5, most preferably 4.1-4.6.

6. The synergistic oral composition as claimed in claim 1, wherein the excipients are selected from the group comprising binder, stabilizing agent, flavoring agent, sweetening agent, viscosity modifying agent, water or any other compound which is not disturbing the pH of the resulted final composition material.

7. The synergistic oral composition as claimed in claim 1, wherein the composition:
i. exhibits high stability at varied temperature range between 6-30° C.,
ii. is semi-viscous form and retained for 2-3 minutes in the Buccal cavity,
iii. mixes with the saliva in 5 to 6 minutes, flow rate of saliva is increased by 10-12 times and it remained higher 2.7 times more when compared to the control group,
iv. the pH of the saliva is also dropped to 4.7 from 7.2 and remained at 4.7 for more than 96 hours.

8. The synergistic oral composition as claimed in claim 1 for use in enhancing the conception rate in dairy animals.

9. A process for the preparation of the synergistic oral composition as claimed in claim 1, wherein the said process comprises the following steps of:
i. Mixing of Amino acid and curcuminoid or its derivative with water to prepare a mixture,
ii. Mixing of both polyhydric alcohol and buffer agent in water to obtain a mixture,
iii. Blending of mixtures from step (i) and (ii) together with pharmaceutically acceptable excipients,
iv. Evaporating excess water under vacuum to yield the composition in the semi-viscous form.

10. The synergistic oral composition as claimed in claim 1, when orally administering to the bovine once 24 to 48 hours before natural or artificial insemination in a dose of between 0.2-1.0 g/kg body weights of the subjected bovine.

11. A method of increasing the sex ratio in a bovine, said method comprising orally administering to the bovine once the composition as claimed in claim 1 in a dose between 0.2-1.0 g/kg body weights, during 24 to 48 hours before natural or artificial insemination of the subject bovine.

12. The method as claimed in claim 11, where the dose up to 320 grams can be administered to a bovine of 600 Kg.
